(19) United States
(12) Patent Application Publication
(10) Pub. No.: US 2007/0292534 A1
(43) Pub. Date: Dec. 20, 2007

(54) Antacid and breath freshening composition

(76) Inventors: Dennis Nelson, Mountain Lakes, NJ (US); Anthony Bell, Andover, NJ (US); Albert Sorg, Columbia, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/810,833

(22) Filed: Jun. 6, 2007

Related U.S. Application Data
(60) Provisional application No. 60/813,894, filed on Jun. 15, 2006.

(51) Int. Cl. A61K 33/24; A61K 33/06; A61K 31/724

(52) U.S. Cl. 424/653; 424/682; 514/58

(57) ABSTRACT

An antacid and breath freshening composition comprising a dose effective antacid, and uncomplexed cyclodextrin is provided. The cyclodextrin may be selected from the group containing alpha-cyclodextrin, beta-cyclodextrin, gamma-cyclodextrin, derivatives thereof and mixtures thereof. The composition is used for the concurrent treatment of various gastro-intestinal conditions and treatment of meal induced halitosis.

There is further provided a cooling composition which can be used in orally administered compositions containing gastrointestinal actives to provide an additional sensation of relief to the throat and esophagus.

This application claims the benefit of U.S. provisional patent application 60/813,894, filed Jun. 15, 2006.

### FIELD OF THE INVENTION

The invention relates to an antacid and breath freshening composition, and to a method of treating gastrointestinal conditions and meal induced halitosis by orally administering the composition. The invention further relates to a cooling composition which can be used in orally administered compositions containing gastrointestinal actives.

### BACKGROUND OF THE INVENTION

The present invention relates generally to compositions comprising active agents useful in the treatment of upper gastrointestinal disorders such as what is commonly referred to as sour stomach, upset stomach, heartburn, gas, acid indigestion and the like. These disorders often occur after consumption of a meal wherein excess acid build up will occur in the stomach and/or will back up into the esophageous from the stomach.

Antacid compositions containing active agents useful for treating gastrointestinal problems are widely used and have been around for quite some time. Ideally, an antacid active provides both rapid and long-lasting relief from the discomfort associated with excess stomach acid. Antacids work by neutralizing or buffering a portion of the gastric hydrochloric acid so that the pH of the stomach is increased from 1 to 2, 3 or 4. Due to this increase in the pH value the symptoms typical of hyperacidity, for example, a feeling of fullness or heartburn, are reduced or even eliminated.

The active ingredients of commercially available, over-the-counter antacid compositions vary and, in general, include metal (e.g., calcium and magnesium) carbonates and hydroxides. More specifically, calcium carbonate is the primary acid neutralizing compound common to many commercially available antacid formulations (e.g., ROLAIDS®, TUMS®). To counteract its constipative effect, calcium carbonate is often used in combination with magnesium salts such as magnesium carbonate, magnesium hydroxide, and magnesium oxide.

Foods that cause gastrointestinal distress, especially heartburn, often have an accompanying offensive odor or malodor, or create an offensive odor through the digestive process thus resulting in a condition referred to as meal induced halitosis. Examples of foods providing or creating strong malodors include certain kinds of liquors, spicy and fatty foods, garlic, fish and certain vegetables such as onions and green peppers, fast foods, pizza, hamburgers, hot dogs, and the like. These strong malodors are apt to remain in the breath for a long time after ingestion of the foods whether trapped in the mouth or trapped in the stomach.

Conventional breath freshening agents are used to mask malodors associated with food intake. For example, ALTOIDS®, CERTS®, BREATH SAVERS®, CLORETS® are available branded products. These confectionery products generally comprise fragrances as their active components which essentially work by masking the malodors generally by one of two mechanisms, either by blending with the malodor to create a different odor or by employing the masking fragrance in a quantity sufficient to overwhelm the malodor. The organic molecules responsible for these strong, unpleasant odors are not removed by these products, negating in-part the effectiveness of the fragrances or masking agents. That is, as the concentration of the fragrance decreases with time the unpleasant odors return.

Cooling agents are often used in conjunction with orally dissolving dosage forms containing gastrointestinal actives, such as antacids, to provide an immediate cooling sensation to the heartburn sufferer. These agents are usually effective in the oral cavity where consumption of the product occurs. The person seeking relief from gastrointestinal distress however further seeks immediate relief of the burning sensation throughout the entire digestive system including relief in the throat and the esophageal area.

There is a need to provide an oral composition having the benefit of providing relief from gastrointestinal distress caused by the consumption of food and attacking the malodor related to the foods that cause the gastrointestinal distress. There is a need for an antacid product which removes rather than masks malodors associated with food induced gastrointestinal distress. There is a need to provide an oral composition which provides the benefit of an immediate sensation of relief to the throat and esophageal area from the heartburn associated with gastrointestinal distress.

### SUMMARY OF THE INVENTION

The invention is directed to an oral composition comprising a dose effective antacid and uncomplexed cyclodextrin. The invention is further directed to a method of treating gastrointestinal conditions and meal induced halitosis comprising orally administering a composition comprising a dose effective antacid and uncomplexed cyclodextrin.

The invention is further directed to an oral composition comprising a gastrointestinal active and a cooling composition.

### DETAILED DESCRIPTION OF THE INVENTION

The invention is directed to an oral composition comprising a dose effective antacid and uncomplexed cyclodextrin. The dose effective antacid comprises one or more acid-neutralizing compounds selected from a group which includes antacids generally known in the art. The uncomplexed cyclodextrin effectively reduces unwanted or unpleasant odors resulting from various compounds present in the mouth or upper gastrointestinal tract.

The antacid may be selected from, but not limited to, sodium bicarbonate, sodium citrate, sodium tartrate, sodium acetate, sodium carbonate, sodium polyphosphate, sodium pyrophosphate, disodium hydrogen phosphate, trisodium phosphate, and other sodium salts, potassium bicarbonate, potassium polyphosphate, potassium pyrophosphate, dipotassium hydrophosphate, tripotassium phosphate, potassium metaphosphate, and other potassium salts, magnesium hydroxide, magnesium lactate, magnesium gluconate, magnesium oxide, magnesium carbonate, magnesium silicate, magnesium trisilicate, magnesium aluminosilicates, and other magnesium salts, aluminum hydroxide, aluminum carbonate, aluminum phosphate, aluminum magnesium hydroxide and other aluminum salts, calcium carbonate, calcium acetate, calcium phosphate, calcium glycerophosphate, calcium chloride, calcium hydroxide, calcium lactate, calcium bicarbonate and other calcium salts, bismuth aluminate, bismuth subsalicylate, bismuth carbonate, bismuth subcarbonate, bismuth subgallate, bismuth subnitrate, and other bismuth salts, and mixtures thereof. A particular embodiment includes an antacid selected from the group consisting of metal bicarbonates, carbonates, hydroxides and oxides and mixtures thereof.

The term “dose effective antacid” means that a person is instructed to administer sufficient antacid to provide 5 mEq or greater of theoretical acid-neutralizing capacity (ANC). For example, if calcium carbonate was the lone acid-neutralizing compound in the antacid, the dose effective antacid would consist of 275 mg or greater of calcium carbonate. This weight calculation is based upon the USP monograph for calcium carbonate: ANC (CaCO₃)=0.9(0.02*X), where X is the quantity in mg of calcium carbonate.

USP monographs for other acid-neutralizing compounds are known to those of ordinary skill in the art. The ANC for aluminum hydroxide is 0.55(0.0385*X) and the ANC for magnesium hydroxide is 0.8(0.0343*X). Again, X is the quantity in mg of the corresponding acid-neutralizing compound. (USP/NF No26).

The oral composition of the present invention may contain from about 5 mEq to about 200 mEq by weight of antacid.

As used herein, the term “cyclodextrin” includes any of the known cyclodextrins such as cyclodextrins containing from six to twelve glucose units, particularly alpha-cyclodextrin, beta-cyclodextrin, gamma-cyclodextrin and their derivatives and mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior, or cavity, of a specific volume. The unique shape and physical-chemical properties of the cavity enable the cyclodextrin molecules to absorb, or form inclusion complexes, with organic molecules or parts of organic molecules including malodor compounds that fit into the cavity. A mixture of cyclodextrins can be used to absorb a range of organic molecules that vary in molecular size.

The term “uncomplexed cyclodextrin” refers to cyclodextrin with essentially unfilled cavities (with the exception of water) prior to the oral administration of the composition.

The term “cyclodextrin” includes any derivatives of cyclodextrin which consist mainly of molecules wherein some of the OH groups are converted to OR groups, wherein R is a substituted or unsubstituted alkyl (alkenyl, cycloalkyl or aryl, etc.) preferably methyl or an ethyl group. Cyclodextrin derivatives include those with short chain alkyl groups such as methylalated cyclodextrins, ethylated cyclodextrins as well as those with hydroxyalkyl substituted groups such as hydroxypropyl cyclodextrins and hydroxyethyl cyclodextrins.

For example, of the seven cyclic glucopyranose units in beta-cyclodextrin three hydroxyl groups in the 2-, 3- and 6-positions can be etherified. In the case of the partially etherified cyclodextrin derivatives, only some of these positions are substituted with hydroxyethyl or hydroxypropyl groups. Thus, hydroxypropyl beta-cyclodextrin is a chemically modified cyclodextrin consisting essentially of an amorphous isomeric mixture of geometric and optical isomers with varying degrees of substitution and varying numbers of hydroxypropyl substituents.

The amount of cyclodextrin in the composition can be from about 5% to about 80% by weight. An embodiment will contain from about 10% to about 50% by weight, cyclodextrin. The term “by weight” is based on the dry weight of the composition excluding the amount of water that is typically added to facilitate the mixing and processing of the composition. The amount of cyclodextrin in the composition will vary within the stated range depending upon the formulation, strength of the antacid and the acid-neutralizing compounds present in the composition.

The amount of cyclodextrin may also depend on the presence of hydrophobic materials in the formulation. Cyclodextrin may complex with these substances upon formulation. Such materials are usually present in most minor amounts. For example, cyclodextrin may complex with hydrophobic flavor oils. The amount of cyclodextrin used should be sufficient to take into account the possible complexation and provide effective malodor control.

The antacids present in the composition can be one or more of known acid-neutralizing compounds. A particular embodiment includes an antacid selected from the group consisting of metal bicarbonates, carbonates, hydroxides and oxides or mixtures thereof. For example, calcium carbonate or magnesium carbonate, can be used alone or in the presence with another antacid agent such as a metal hydroxide such as magnesium hydroxide, aluminum hydroxide, and aluminum-magnesium hydroxide or can be used with other acid neutralizing compounds such as magnesium oxide or calcium oxide.

The composition may contain a ratio by weight of the cyclodextrin to the antacid of from about 1.0/0.1 to about 1.0/4.5. An embodiment may contain a ratio of from about 1/1.5 to ¼.

The oral compositions of the invention are ingestible and digestible oral delivery compositions. The compositions may effectively work in the oral cavity and in the upper gastrointestinal tract or stomach. The composition may complex malodors in the mouth as well as malodors forming in the digestive tract as needed.

An aspect of the invention includes the presence of a particular cooling composition in the oral composition. The cooling composition is useful in any orally dissolvable dosage form containing actives for gastrointestinal relief such as antacids, Histamine₂ Receptor Antagonists (H₂RAs) Proton Pump Inhibitors (PPIs) and the like. In that it provides immediate cooling to the areas of the throat and the upper digestive tract, it is particularly useful in compositions, such as those containing antacids, wherein the sufferer seeks immediate relief of the burning sensation produced in those areas.

The composition consists essentially of a combination of (a) menthol (b) one or more non-mentholic cooling agents and (c) a cooling sugar alcohol. Non-mentholic cooling agents are commonly known in the art. Examples of non-mentholic cooling agents are WS-23, WS-3, MPD, Physcool, Coolenol, Frescolat and the like. Sugar alcohols are commonly known in the art. Examples of cooling sugar alcohols include xylitol, erythritol, mannitol, sorbitol, lactitol and the like. Menthol is understood to include all forms thereof. In an embodiment the sugar alcohol is xylitol. A particular embodiment uses the combination of menthol, WS-23 and xylitol.

The cooling composition is composed by weight of (a) menthol in the range of about 0.05 to about 10%, (b) one or more non-mentholic cooling agents in the range of about 0.1 to about 20% and (c) sugar alcohol in the range of about 70 to about 99.85%. In an embodiment the (a) menthol is from about 1.0 to about 5%, the (b) one or more non-mentholic cooling agents is from about 2 to about 10% and the (c) sugar alcohol is from about 85 to about 97%.

The cooling composition may be incorporated into the orally dissolvable dosage form at a total concentration of from about 3% to about 40% by weight. In an embodiment the concentration is from about 5% to about 30% by weight.

The oral composition is typically provided in the form of chewable and/or orally dissolvable compositions. Suitable forms include chewable pressed tablets, soft chews, fast-melt tablets, lozenges, dissolvable tablets, powders and the like. In one aspect the dosage form is a chewable pressed tablet. In another aspect the dosage form is a soft chew format. Other comestibles known to those of ordinary skill containing the described components of the described composition can also be used. These comestibles are made according to methods well known in the art.

Compressed tablets contain particular materials and are formed into structures under pressure. These compositions generally contain typical tablet excipients such as binders and lubricants as well as sweeteners, flavoring agent, colorants and so forth. Cyclodextrin has properties which allow it to act as a compression powder, therefore, in compressed tablet formulations, cyclodextrin can be used in part to replace tabletting aids such as cellulose. These confections may sugared or sugarless. The tablet may also be coated or uncoated. Tablet size may range from 0.1 g to 6.0 g. Larger tablets may be formulated for particular targets.

Exemplary of soft chew formats are caramel, fudge and nougat and the like. These compositions generally contain as components, fats, carbohydrates (including sugar, glucose or corn syrup and sugar substitutes), milk/protein, flavors, and excipients such as emulsifiers, aerating agents, hydrocolloids, colors and the like. They are made by processes generally known in the art. A general discussion of the composition and preparation of soft chew confections may be found in E. B. Jackson, Ed. “Sugar Confectionery Manufacture”, 2nd edition, Blackie Academic & Professional Press, Glasgow UK, (1990), at pages 170-188.

In one aspect the process comprises preparing a mixture of components, heating the mixture to a temperature sufficient to cook it and form a cooked confectionery composition, and cooling the mixture to form the confectionery composition. Alternatively the ingredients may be cold processed to form the soft chew composition.

The oral composition may contain sweetener. The sweetener used in the compositions can be one or a combination of any known sweeteners. An exemplary list of the more common sugars include corn syrup, lactose, glucose, maltose, sucrose, fructose, dextrose and the like. The sweetener can also be a sugar substitute such as a hydrogenated starch hydrolysate, a sugar alcohol such as sorbitol, erythritol, xylitol, mannitol, lactitol, maltitol and the like, or polydextrose and the like. Cooling sugar alcohols may also be present for their cooling effects. Of course, any type of sweetener other than those listed above can be used alone or in combination

The sweetening effect may also be provided by a high intensity sweetener alone or in combination with other sweeteners. Representative examples are saccharin, aspartame, sodium cyclamate, sucralose, acesulphame K (Ace K), alitame, glycyrrhizin, thaumatin and the like.

The amount of sweetener present in the composition can range up to 95% by weight depending on the delivery system format. Typically compositions may contain from 0.05% to 40% sweetener by weight. In soft chew formats the amount of sweetener may be from 0.05% to 80% by weight. High intensity sweeteners may generally be present in amounts of from about 0.05 to 2.0% by weight.

The oral composition can also contain a flavoring agent. Flavoring agents include essential oils as well as various flavoring aldehydes, esters, alcohols and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. The flavoring agent may be incorporated in the oral composition at a concentration of about 0.5% to about 5% by weight.

Solid flavoring agents can have certain advantages over liquid (oil) flavoring agents because they tend not to form inclusion complexes with the cyclodextrins. Solid flavoring agents are either solids per se or oils usually combined with solid platforms such as resins, maltodextrins and the like. Examples of these are spray or freeze dry formats.

Cooling agents may be used per se in the composition alone or in combination. The cooling agent(s) may generally be incorporated at a concentration of from about 0.1% to about 4% by weight.

A particular composition used for the treatment of various gastro-intestinal conditions will comprise a metal carbonate, uncomplexed cyclodextrin, a flavoring agent and a sweetener. An alternate composition will further comprise the cooling composition of the present invention.

The oral compositions can be used for the treatment of various upper gastrointestinal disorders such as what is commonly referred to as sour stomach, upset stomach, heartburn, gas, acid indigestion and the like. The compositions are provided in a form suitable for oral administration.

The method comprises orally administering to a human a safe and effective amount of the antacid in combination with cyclodextrin. As used herein, the term “safe and effective amount” refers to a quantity of the antacid sufficient to provide 5 mEq or greater of acid neutralizing capacity without undue adverse side effects such as toxicity, irritation, or allergic response. The specific safe and effective amount will vary with such factors as the specific condition that is being treated, the severity of the condition, the duration of the treatment, the physical condition of the subject, the nature of any concurrent therapy, and the specific acid-neutralizing compound in the antacid composition. A human patient in need of such treatment will typically receive from about 180 mg to about 2,000 mg of acid-neutralizing compound daily.

### EXAMPLES

### Example 1

### Antacid Mix

| Ingredient | % by weight | |
|---|---|---|
| Calcium carbonate | 69.64 | |
| Magnesium hydroxide | 13.65 | |
| Sucrose | 14.71 | |
| Starch | 1.00 | |
| Polyethylene glycol, powdered | 1.00 | |
| Total | 100.00 | g |

The calcium carbonate, magnesium hydroxide, sucrose, powdered polyethylene glycol and starch were added to a ribbon blender. During the mixing 11 g water were added over a five minute period and the mixing continued for additional 45 min. The mixture was then extruded and allowed to dry at about 70° C. The dried extrudate was then milled to provide an antacid mix. The antacid mix was then used as noted in the following examples.

### Examples 2, 3 and 4

Example 2 is a typical chewable antacid tablet formulation. Example 3 illustrates total replacement of the microcrystalline cellulose tabletting aid with uncomplexed beta-cyclodextrin. Example 4 illustrates replacement with a mixture of beta-cyclodextrin and dextrose.

| Ingredient* | Example 2 | Example 3 | Example 4 |
|---|---|---|---|
| Example 1 Antacid Mix | 54.42 | 54.42 | 54.42 |
| Dextrose monohydrate | 0.00 | 0.00 | 20.00 |
| Beta-cyclodextrin | 0.00 | 40.00 | 20.00 |
| Cornstarch | 3.00 | 3.00 | 3.00 |
| Cherry flavor | 2.00 | 2.00 | 2.00 |
| Color | 0.08 | 0.08 | 0.08 |
| Magnesium stearate | 0.50 | 0.50 | 0.50 |
| Microcrystalline cellulose | 40.00 | 0.00 | 0.00 |
| Total | 100.00 | 100.00 | 100.00 |

*Percent by Weight

The antacid mix and remaining ingredients were blended and pressed into tablets. The tablets ranged from 1.8 to 2.0 g.

### Examples 5 and 6

Examples 5 and 6 illustrate further chewable tablets of the invention using the antacid premix. Both contain a mixture of cyclodextrins. Various antacid actives and cooling combinations are used.

| Ingredient* | Example 5 | Example 6 |
|---|---|---|
| Example 1 Antacid Mix | 45.81 | 64.32 |
| Aluminum hydroxide | 0.00 | 7.31 |
| Magnesium stearate | 0.40 | 0.20 |
| Stearic Acid | 2.45 | 1.25 |
| Sucralose | 0.23 | 0.12 |
| Gamma-cyclodextrin | 9.48 | 5.85 |
| Beta-cyclodextrin | 9.48 | 5.85 |
| Alpha-cyclodextrin | 9.20 | 5.85 |
| Mint Flavors | 2.30 | 1.16 |
| Creamy Mint Flavors | 1.35 | 0.69 |
| WS-23 Cooling Agent | 0.68 | 0.35 |
| Stabilok SD Menthol | 0.40 | 0.20 |
| Xylitab (xylitol) | 18.22 | 6.85 |
| Total | 100.00 | 100.00 |

*Percent by Weight

In Example 5 all of the ingredients but for the cyclodextrins, magnesium stearate and creamy peppermint were mixed and blended for 2 minutes in a suitable blender with the creamy peppermint added while blending. The cyclodextrins and magnesium stearate were then added and blended for 3 minutes. The mixture was compressed on a Carver press at various compression forces ranging from 2500 lbs to 4500 lbs. The tablets were formed at 1.74 gms.

Example 6 is similarly prepared.

### Examples 7, 8, 9, 10

Examples 7, 8, 9 and 10 illustrate sugar-free chewable tablets of the invention. Various antacid actives, combinations of cyclodextrins, and cooling combinations are used.

| Ingredient* | Example 7 | Example 8 | Example 9 | Example 10 |
|---|---|---|---|---|
| Calcium Carbonate | 60.73 | 40.55 | 37.29 | 55.15 |
| Magnesium hydroxide | 0.00 | 8.11 | 7.46 | 0.00 |
| Aluminum hydroxide | 0.00 | 0.00 | 0.00 | 9.19 |
| Magnesium stearate | 0.28 | 0.33 | 0.44 | 0.26 |
| Stearic Acid | 1.72 | 2.22 | 2.04 | 1.57 |
| Sucralose | 0.16 | 0.29 | 0.27 | 0.15 |
| Ethyl Maltol | 0.00 | 0.00 | 0.05 | 0.00 |
| Gamma-cyclodextrin | 8.10 | 12.02 | 0.00 | 7.35 |
| Beta-cyclodextrin | 8.10 | 12.02 | 3.32 | 7.35 |
| Alpha-cyclodextrin | 8.10 | 12.02 | 7.74 | 7.35 |
| Avicel PH 102 | 0.00 | 0.00 | 9.71 | 0.00 |
| Chlorophyll | 0.00 | 0.00 | 0.06 | 0.00 |
| Mint Flavors | 1.62 | 2.28 | 0.00 | 1.46 |
| Creamy Mint Flavors | 0..95 | 1.80 | 1.20 | 0.87 |
| Robertet Art. Cooling | 0.00 | 0.00 | 0.46 | 0.00 |
| Enhancer Powder** | | | | |
| Vanilla Flavor | 0.00 | 0.26 | 0.00 | 0.00 |
| WS-23 Cooling Agent | 0.48 | 0.02 | 0.00 | 0.43 |
| Coolenol SD | 0.00 | 0.39 | 0.14 | 0.00 |
| Stabilok SD Menthol | 0.28 | 1.95 | 0.00 | 0.26 |
| Xylitab (xylitol) | 9.48 | 5.74 | 29.82 | 8.61 |
| Total | 100.00 | 100.00 | 100.0 | 100.00 |

*Percent by Weight
**Cooling agent mixture which includes menthol and non-mentholic agents

Examples 7, 8 and 9 were similarly prepared. In Example 7 all of the ingredients but for the cyclodextrins, magnesium stearate stearic acid and creamy peppermint were mixed and blended for 2 minutes in a suitable blender with the creamy peppermint added while blending. The cyclodextrins, stearic acid and magnesium stearate were then added and blended for 3 minutes. In Examples 8 and 9 all of the ingredients but for the magnesium stearate were mixed and blended, for 2 minutes in Example 8 and 3 minutes in Example 9, with the magnesium stearate separately added and blended for 3 minutes. The mixtures were both compressed on a Carver press at various compression forces ranging from 2500 lbs to 4500 lbs for Examples 7 and 8 and from 1300 lbs to 5000 lbs for Example 9. The Example 7 and 8 tablets were formed at approximately 1.7 gms and Example 9 at 1.8 gms.

Example 10 is similarly prepared.

### Examples 11 and 12

Examples 11 and 12 illustrate soft chew compositions of the invention. Combinations of cyclodextrins and various antacid actives are used.

| Ingredient* | Example 11 | Example 12 |
|---|---|---|
| Calcium Carbonate, precipated | 22.32 | 21.31 |
| Aluminum hydroxide | 0.00 | 4.53 |
| Confectioner's sugar | 23.81 | 22.73 |
| Corn syrup solids Drisweet 36 | 2.99 | 2.85 |
| Corn syrup Clearsweet 43/43 | 31.30 | 29.88 |
| Gamma-cyclodextrin | 3.79 | 3.62 |
| Beta-cyclodextrin | 3.79 | 3.62 |
| Alpha-cyclodextrin | 3.79 | 3.62 |
| Mint Flavor | 0.95 | 0.91 |
| Vanilla Mint Flavors | 1.04 | 1.00 |
| Sweet Cream Flavor | 0.05 | 0.04 |
| Glycerine | 0.95 | 0.91 |
| Hydrogenated Coconut oil | 5.01 | 4.78 |
| Lecithin | 0.21 | 0.20 |
| Total | 100.00 | 100.00 |

*Percent by Weight

The coconut oil and lecithin are premixed at 110° F. with stirring to form a fat mix. The solids are separately mixed and blended for 5 minutes. The corn syrup is separately heated to 110° F. The liquids (flavors, glycerine, fat mix, corn syrup) are added to the solids and mixed for an additional 5 minutes until uniform. The product is formed into piece shapes.

### Example 13

### Evaluations

The tablets prepared in Example 7 were used in evaluations as follows:

Odor Absorption:
A garlic and onion oil solution was prepared. A crushed tablet was added with stirring to a portion of the solution in a beaker. A panel of three evaluated the odor of both solutions. The panelists found the inventive example to have an acceptable reduction in the garlic and onion odors as compared to the control.

Cooling Sensation:
A panel of four chewed and swallowed prepared tablets. This panel found a strong cooling sensation to be delivered not only to the mouth but also to the throat/upper esophagus region.

We claim:

1. An oral composition comprising a dose effective antacid and uncomplexed cyclodextrin.

2. The composition according to claim 1 wherein the antacid comprises an antacid selected from the group consisting of metal bicarbonates, carbonates, hydroxides and oxides and mixtures thereof.

3. The composition according to claim 1 wherein the antacid comprises an antacid selected from the group consisting of sodium bicarbonate, sodium citrate, sodium tartrate, sodium acetate, sodium carbonate, sodium polyphosphate, sodium pyrophosphate, disodium hydrogen phosphate, trisodium phosphate, other sodium salts, potassium bicarbonate, potassium polyphosphate, potassium pyrophosphate, dipotassium hydrophosphate, tripotassium phosphate, potassium metaphosphate, other potassium salts, magnesium hydroxide, magnesium lactate, magnesium gluconate, magnesium oxide, magnesium carbonate, magnesium silicate, magnesium trisilicate, magnesium aluminosilicates, other magnesium salts, aluminum hydroxide, aluminum carbonate, aluminum phosphate, aluminum magnesium hydroxide, other aluminum salts, calcium carbonate, calcium acetate, calcium phosphate, calcium glycerophosphate, calcium chloride, calcium hydroxide, calcium lactate, calcium bicarbonate, other calcium salts, bismuth aluminate, bismuth subsalicylate, bismuth carbonate, bismuth subcarbonate, bismuth subgallate, bismuth subnitrate, other bismuth salts and mixtures thereof.

4. The composition according to claim 1 wherein the cyclodextrin is selected from the group consisting of alpha-cyclodextrin, beta-cyclodextrin, gamma-cyclodextrin and derivatives thereof and mixtures thereof.

5. The composition according to claim 1 which is an ingestible and digestible delivery system.

6. The composition according to claim 5 which is selected from the group consisting of chewable pressed tablets, soft chews, fast-melt tablets, lozenges and dissolvable tablets.

7. The composition according to claim 1 wherein the antacid is present in an amount of from about 5 mEq.

8. The composition according to claim 1 wherein the cyclodextrin is present in an amount of from about 5% to 80% by weight.

9. A method of treating conditions associated with gastrointestinal disorders and meal induced halitosis comprising administration of an effective amount of an oral composition comprising a dose effective antacid and uncomplexed cyclodextrin.

10. The method according to claim 9 wherein the dose effective antacid agent comprises an antacid selected from the group consisting of metal bicarbonates, carbonates, hydroxides and oxides and mixtures thereof.

11. The method according to claim 9 wherein the dose effective antacid agent comprises an antacid selected from the group consisting of sodium bicarbonate, sodium citrate, sodium tartrate, sodium acetate, sodium carbonate, sodium polyphosphate, sodium pyrophosphate, disodium hydrogen phosphate, trisodium phosphate, other sodium salts, potassium bicarbonate, potassium polyphosphate, potassium pyrophosphate, dipotassium hydrophosphate, tripotassium phosphate, potassium metaphosphate, other potassium salts, magnesium hydroxide, magnesium lactate, magnesium gluconate, magnesium oxide, magnesium carbonate, magnesium silicate, magnesium trisilicate, magnesium aluminosilicates, other magnesium salts, aluminum hydroxide, aluminum carbonate, aluminum phosphate, aluminum magnesium hydroxide, other aluminum salts, calcium carbonate, calcium acetate, calcium phosphate, calcium glycerophosphate, calcium chloride, calcium hydroxide, calcium lactate, calcium bicarbonate, other calcium salts, bismuth aluminate, bismuth subsalicylate, bismuth carbonate, bismuth subcarbonate, bismuth subgallate, bismuth subnitrate, other bismuth salts and mixtures thereof.

12. The method according to claim 9 wherein the cyclodextrin is selected from the group consisting of alpha-cyclodextrin, beta-cyclodextrin, gamma-cyclodextrin and derivatives thereof and mixtures thereof.

13. The method according to claim 9 wherein the oral composition is an ingestible and digestible oral delivery confectionery composition.

14. The method according to claim 13 which is selected from the group consisting of chewable pressed tablets, soft chews, fast-melt tablets, lozenges and dissolvable tablets.

15. The method according to claim 9 wherein the antacid is present in an amount of from about 5 mEQ.

16. The method according to claim 9 wherein the cyclodextrin is present in an amount of from about 5% to 80% by weight.

17. An orally consumable composition comprising
(1) one or more gastrointestinal actives and
(2) a cooling composition consisting essentially of
(a) menthol present in an amount of about 0.05 to about 10% by weight,
(b) one or more non-mentholic cooling agent present in an amount of about 0.1 to about 20% by weight, and
(c) a cooling sugar alcohol present in the amount of about 70 to about 99.85% by weight.

18. The oral composition of claim 17 wherein the cooling composition is present in an amount from about 3% to about 40% by weight of the oral composition.

19. The oral composition of claim 17 wherein the active is an antacid.

20. The oral composition of claim 19 further comprising uncomplexed cyclodextrin.

21. A method of providing a cooling effect to the throat and esophagus by administering the orally consumable composition of claim 17.

22. The method of claim 21 wherein the cooling composition is present in an amount from about 3% to about 40% by weight of the oral composition.
